(19) United States
(12) Patent Application Publication
(10) Pub. No.: US 2021/0255111 A1
(43) Pub. Date: Aug. 19, 2021

(54) METHODS AND DEVICES FOR PERFORMING AN ANALYTICAL MEASUREMENT

(71) Applicant: Roche Diabetes Care, Inc., Indianapolis, IN (US)

(72) Inventors: Bernd Limburg, Soergenloch (DE); Max Berg, Mannheim (DE); Fredrik Hailer, Mannheim (DE)

(21) Appl. No.: 17/308,730

(22) Filed: May 5, 2021

Related U.S. Application Data
(63) Continuation of application No. PCT/EP2019/080154, filed on Nov. 5, 2019.

(30) Foreign Application Priority Data
Nov. 7, 2018 (EP) 18 205 002.1

(51) Int. Cl. G01N 21/78; G06T 7/90; H04N 5/225; G06T 7/00

(52) U.S. Cl. CPC G01N 21/78; G06T 7/90; G06T 2207/10024; G06T 7/0014; H04N 5/2256

(57) ABSTRACT

A method of performing an analytical measurement based on a color formation reaction in an optical test strip using a mobile device having a camera. In the inventive method, an optical test strip having a test field is provided. A first image of at least part of the test field is captured using the camera with an image acquisition setting of the camera, and the image acquisition setting is locked. A sample of body fluid is applied to the test field and then there is a step of waiting a predetermined minimum amount of time. Then, while the image acquisition setting remains locked, a second image of at least part of the test field is captured using the camera with the same image acquisition setting of the camera used to capture the first image. An analytical measurement result value is then determined by using the first and the second images.

### RELATED APPLICATIONS

This application is a continuation of PCT/EP2019/080154, filed Nov. 5, 2019, which claims priority to EP 18 205 002.1, filed Nov. 7, 2018, the entire disclosures of both of which are hereby incorporated herein by reference.

### BACKGROUND

The present application refers to a method of performing an analytical measurement based on a color formation reaction in an optical test strip by using a mobile device having a camera. This disclosure further relates to computer programs for executing the method according to this disclosure. Further, this disclosure refers to a mobile device and a kit for performing an analytical measurement. Methods, computer programs, mobile devices and kits according to this disclosure may be used in medical diagnostics, in order to for example qualitatively or quantitatively detect one or more analytes in one or more body fluids. Other fields of application of this disclosure, however, are feasible.

In the field of medical diagnostics, in many cases, one or more analytes have to be detected in samples of a body fluid, such as blood, interstitial fluid, urine, saliva or other types of body fluids. Examples of analytes to be detected are glucose, triglycerides, lactate, cholesterol or other types of analytes typically present in these body fluids. According to the concentration and/or the presence of the analyte, an appropriate treatment may be chosen, if necessary. Without narrowing the scope, this disclosure specifically may be described with respect to blood glucose measurements. It shall be noted, however, that this disclosure may also be used for other types of analytical measurements using test elements.

Generally, devices and methods known to the skilled person make use of test elements comprising one or more test chemicals, which, in presence of the analyte to be detected, are capable of performing one or more detectable detection reactions, such as optically detectable detection reactions. As an example, EP 0 821 234 A2 describes a diagnostic test carrier for the determination of an analyte from whole blood with the aid of a reagent system contained in the carrier and a method for the determination of an analyte from whole blood with the aid of the diagnostic test carrier. The diagnostic test carrier includes a color forming reagent. The test field has a sample application side to which the blood sample is delivered and a detection side where an optically detectable change occurs as a result of the reaction of the analyte with the reagent system. Furthermore, the test field is designed so that the erythrocytes contained in the sample do not reach the detection side. In addition, the test field comprises a transparent film and a first and a second superposed film layer applied thereto, wherein the first layer on the transparent film is substantially less light-scattering in the wet state than the overlying second layer.

With regard to the test chemicals comprised in test elements, reference may be made, e.g., to J. Hoenes et al.: The Technology Behind Glucose Meters: Test Strips, Diabetes Technology & Therapeutics, Volume 10, Supplement 1, 2008, S-10 to S-26. Other types of test chemistry are possible and may be used for performing this disclosure.

Typically, one or more optically detectable changes in the test chemical are monitored, in order to derive the concentration of the at least one analyte to be detected from these changes. In general, variable lighting conditions need to be taken into account. Thus, as an example, EP 2 916 117 Al discloses color quantification of chemical test pads and titration of analytes which can be performed under different lighting conditions. In one embodiment, the lighting condition under which a digital image is captured is estimated and utilized to select a set of reference colors for determining the titration. In another embodiment, a plurality of comparisons is made under different lighting conditions and the result having the highest confidence level is selected for determining the titration.

Further, WO 2014/037462 A1 discloses a method and a device for determining sample application on an analytical test element in a photometric reflectance measuring device specifically for glucose measurements, where the following measures are proposed: providing a disposable test element for application of a body fluid sample, taking a sequence of reflectance readings from the test element, monitoring a change of the reflectance readings with respect to a sample application condition, adjusting the sample application condition in accordance with a drift correction. A value for drift correction of the reflectance readings caused by ambient measurement conditions, specifically, humidity, temperature, or UV radiation can be considered in a predefined signal decrease or in a predefined signal threshold for adjusting the sample application condition.

For simple and reliable control of the suitability for use of analytical elements, EP 1 189 064 A1 proposes to calculate the variance of the quotient from a control value and a first standard reference value with respect to a first reference quotient which is from a control reference value and the first standard reference value.

EP 0 953 149 B1 discloses a method of analysis of image data from a chemical diagnostic assay which assay generates an image result on a substrate. The method comprising the steps of: i) obtaining a said image result on a substrate; and ii) imaging the image result with an image acquisition device to generate digital color image data corresponding to the color composition and distribution of the image result; and iii) using data processing means, applying to the digital color image data a stored relationship between the color composition of the image result and assay calibration data to generate a quantified result for said assay. Further, said assay calibration data comprises a set of calibration values for combinations of at least two selected color parameters; and said quantified result is generated by interpolation between at least two of said calibration values.

U.S. Publication No. 2017/0098137 A1 describes a reagent test paddle including a contamination detection medium, a reference color bar, at least one chemical test medium, and a unique identifier. The contamination detection medium includes a reagent that changes color in the presence or when exposed to a hostile or inhospitable environment. Each chemical test medium includes a regent that is responsive to a respective analyte in a biological sample. The reference color bar includes reference color samples of different colors. The unique identifier, like a serial number, identifies the particular paddle and its chemical test medium so it can be uniquely and anonymously associated with a user.

WO 2014/025415 A2_describes a method and a device for performing color-based reaction testing of biological materials. The method includes capturing and interpreting digital images of an unexposed and later exposed instrument within an automatically calibrated environment. The instrument includes a Unique Identification (UID) label, Reference Color Bar (RCB) providing samples of standardized colors for image color calibration, and several test-specific sequences of Chemical Test Pads (CTP). The method further includes locating the instrument in the image, extracting the UID, extracting the RCB, and locating the plurality of CTP in each image. The method further reduces image noise in the CTP and calibrates the image automatically according to lighting measurements performed on the RCB. The method further determines test results by comparing the color of the CTP image to colors in a Manufacturer Interpretation Color Chart (MICC). The method shows these results in graphical or quantified mode.

U.S. Publication No. 2013/0267032 A1_describes a specimen test strip to detect a characteristic of an analyte in a specimen sample. The specimen test strip includes a reaction area to receive the specimen sample and a color calibration area to determine a color, or a color and a color intensity, of the reaction area after receiving the specimen sample. The specimen test strip may further include a temperature indication area to correct a measurement of the characteristic of analyte.

U.S. Publication No. 2015/0233898 A1 described a test strip module including a case, and a position anchor extending down past a mating surface to a face of a mobile computing device. The position anchor has a shape matching a feature on the face of the mobile computing device.

U.S. Publication No. 2012/0329170 A1 describes an analyzing apparatus containing an image pickup unit and an analysis unit. The image pickup unit contains a view field area that covers at least a reaction area and a background area in a test piece. The reaction area exhibits a reaction color when exposed to a test substance in a specimen. The analysis unit detects the test substance based on the reaction color and determines, during detection of the test substance, whether a state of the background area falls within an acceptable range set for the test substance.

WO 2017/059103 A1 describes a method that includes capturing a signal of a detection substrate exposed to a sample containing a target substance; determining that the detection substrate is in a testable state; and generating an assessment of the presence of the target substance in the sample.

DE 10 2016 202 428 A1 describes a measuring system for carrying out colorimetric assays and a corresponding method with this measuring system comprising at least one test strip having at least one test field for determining a concentration of at least one analyte in a test solution by means of a color change of the test field, wherein each test strip is associated with a machine-readable code with data for identification of the test strip and for evaluation by an application, a mobile device with a camera for executing a first image of at least the machine-readable code before contact of the test strip with the test solution and a second image of the test strip after contact with the test solution and with the application for evaluating the first and second recordings installed on the mobile device, the application at least is seen on the basis of read on the first recording machine-readable code to verify the usability of the test strip for each measurement automatically and in the case of usability for this test strip associated colorimetric Assay and associated calibration data automatically for the evaluation of the color change of the test strip set, and only then by means of the second image using the colorimetric assay automatically the color change of the test fields or and calculate the concentration of the analyte based on the calibration data.

Besides using customized detectors which are specifically developed for the purpose of optically detecting changes in the test chemistry comprised by corresponding test elements, recent developments aim at using widely available devices such as smartphones. However, when using consumer-electronics having a camera, such as smartphones, individual technical and optical properties may have to be taken into account, since there is a vast number of cameras available on the market, which may have an impact on the determination of the analyte concentration.

Thus, despite the advantages involved in using consumer-electronics having a camera for the purpose of detecting an analyte in a sample or evaluating analytical measurements, several technical challenges remain. In general, color representation in camera systems is adapted to provide images which are optimized in terms of human color perception, e.g., by internal post-processing of the raw data captured by the camera. However, such post-processing due to human color perception may not be ideal when aiming at accurately determining analyte concentrations in the sample.

### SUMMARY

This disclosure teaches methods and devices which address the above-mentioned technical challenges of analytical measurements using mobile devices such as consumer-electronics mobile devices, specifically multipurpose mobile devices which are not dedicated to analytical measurements such as smartphones or tablet computers.

Specifically, methods, computer programs and devices are disclosed herein which are widely applicable to available mobile devices and which are suited to increase measurement accuracy and improve reliability while allowing convenient handling for the user.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “image,” “camera,” and “image acquisition setting,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities.

Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect, a method of performing an analytical measurement based on a color formation reaction in an optical test strip by using a mobile device having a camera is disclosed. The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed.

In general, the method comprises the following steps:
a) providing an optical test strip having a test field without having a sample applied thereto;
b) capturing at least one first image of at least part of the test field of the optical test strip without having a sample applied thereto by using the camera with at least one image acquisition setting, specifically with a set of acquisition settings of the camera;
c) applying a sample, specifically a drop, of bodily fluid to the test field of the optical test strip;
d) waiting for a predetermined minimum amount of time;
e) capturing at least one second image of at least part of the test field of the optical test strip having the sample of bodily fluid applied thereto by using the camera with the one or more image acquisition settings of the camera, wherein the image acquisition settings of the camera are the same image acquisition settings of the camera as used in step b); and
f) determining an analytical measurement result value by using the first and the second image of the optical test field of the optical test strip, specifically by comparing the at least two images.

The term “analytical measurement” as used herein is a broad term and is to be given its ordinary and customary meaning to a person of ordinary skill in the art and is not to be limited to a special or customized meaning. The term specifically may refer, without limitation, to a quantitatively and/or qualitatively determination of at least one analyte in an arbitrary sample. For example, the sample may comprise a bodily fluid, such as blood, interstitial fluid, urine, saliva or other types of body fluids. The result of the analytical measurement, as an example, may be a concentration of the analyte and/or the presence or absence of the analyte to be determined. Specifically, as an example, the analytical measurement may be a blood glucose measurement, thus the result of the analytical measurement may for example be a blood glucose concentration. In particular, an analytical measurement result value may be determined by the analytical measurement. The term “analytical measurement result value” as used herein is a broad term and is to be given its ordinary and customary meaning to a person of ordinary skill in the art and is not to be limited to a special or customized meaning. The term specifically may refer, without limitation, to a numerical indication of an analyte concentration in a sample.

The at least one analyte, as an example, may be or may comprise one or more specific chemical compounds and/or other parameters. As an example, one or more analytes may be determined which take part in metabolism, such as blood glucose. Additionally or alternatively, other types of analytes or parameters may be determined, e.g., a pH value. The at least one sample, specifically, may be or may comprise at least one bodily fluid, such as blood, interstitial fluid, urine, saliva or the like. Additionally or alternatively, however, other types of samples may be used, such as water.

The analytical measurement, specifically, may be an analytical measurement including a change of at least one optical property of an optical test strip, which change may be measured or determined visually by using the camera. Specifically, the analytical measurement may be or may comprise a color formation reaction in the presence of the at least one analyte to be determined. The term “color formation reaction” as used herein is a broad term and is to be given its ordinary and customary meaning to a person of ordinary skill in the art and is not to be limited to a special or customized meaning. The term specifically may refer, without limitation, to a chemical, biological or physical reaction during which a color, specifically a reflectance, of at least one element involved in the reaction, changes with the progress of the reaction.

The term “optical test strip” as used herein is a broad term and is to be given its ordinary and customary meaning to a person of ordinary skill in the art and is not to be limited to a special or customized meaning. The term specifically may refer, without limitation, to an arbitrary element or device configured for performing a color-change detection reaction. The optical test strip may also be referred to as test strip or test element, wherein all three terms may refer to the same element. The optical test strip may particularly have a test field containing at least one test chemical for detecting at least one analyte. The optical test strip, as an example, may comprise at least one substrate, such as at least one carrier, with the at least one test field applied thereto or integrated therein. In particular, the optical test strip may further comprise at least one white area, such as a white field, specifically in a proximity to the test field, for example enclosing or surrounding the test field. The white are may be a separate field independently arranged on the substrate or carrier. However, additionally or alternatively, the substrate or carrier itself may be or may comprise the white area. As an example, the at least one carrier may be strip-shaped, thereby rendering the test element a test strip. These test strips are generally widely in use and available. One test strip may carry a single test field or a plurality of test fields having identical or different test chemicals comprised therein.

As further used herein, the term “test field” is a broad term and is to be given its ordinary and customary meaning to a person of ordinary skill in the art and is not to be limited to a special or customized meaning. The term specifically may refer, without limitation, to a coherent amount of the test chemical, such as to a field, e.g., a field of round, polygonal or rectangular shape, having one or more layers of material, with at least one layer of the test field having the test chemical comprised therein.

The term “mobile device” as used herein is a broad term and is to be given its ordinary and customary meaning to a person of ordinary skill in the art and is not to be limited to a special or customized meaning. The term specifically may refer, without limitation, to a mobile electronics device, more specifically to a mobile communication device such as a cell phone or smartphone. Additionally or alternatively, as will be outlined in further detail below, the mobile device may also refer to a tablet computer or another type of portable computer having at least one camera.

The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, two-dimensional or even three-dimensional optical data or information. As an example, the camera may comprise at least one camera chip, such as at least one CCD chip and/or at least one CMOS chip configured for recording images. As used herein, without limitation, the term “image” specifically may relate to data recorded by using a camera, such as a plurality of electronic readings from the imaging device, such as the pixels of the camera chip.

The camera, besides the at least one camera chip or imaging chip, may comprise further elements, such as one or more optical elements, e.g., one or more lenses. As an example, the camera may be a fix-focus camera, having at least one lens which is fixedly adjusted with respect to the camera. Alternatively, however, the camera may also comprise one or more variable lenses which may be adjusted, automatically or manually. This disclosure specifically shall be applicable to cameras as usually used in mobile applications such as notebook computers, tablets or, specifically, cell phones such as smart phones. Thus, specifically, the camera may be part of a mobile device which, besides the at least one camera, comprises one or more data processing devices such as one or more data processors. Other cameras, however, are feasible.

The camera specifically may be a color camera. Thus, such as for each pixel, color information may be provided or generated, such as color values for three colors R, G, B. a larger number of color values is also feasible, such as four color values for each pixel, for example R, G, G, B. Color cameras are generally known to the skilled person. Thus, as an example, the camera chip may consist of a plurality of three or more different color sensors each, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used, such as R, G, G, B. The color sensitivities of the pixels may be generated by color filters or by appropriate intrinsic sensitivities of the sensor elements used in the camera pixels. These techniques are generally known to the skilled person.

Both steps b) and e) comprise capturing at least one image by using the camera, wherein the camera is used with the at least one image acquisition setting. As used herein, the term “image acquisition setting” is a broad term and is to be given its ordinary and customary meaning to a person of ordinary skill in the art and is not to be limited to a special or customized meaning. The term specifically may refer, without limitation, to an arbitrary configuration or adjustment of an object or element, specifically of an imaging device, e.g., of a camera, when the object or element is used for capturing at least one image or picture. In particular, image acquisition settings may be configurations of the camera, such as the camera of the mobile device, when capturing the at least one image. For example, the image acquisition setting may be one or more of a shutter speed, an exposure time, a lens focus, a color adjustment, a color saturation, a pixel correction, a noise reduction or the like. Thus, when a camera is used for capturing at least one image, the image acquisition settings of the camera may also simply be referred to as image acquisition settings.

In particular, the image acquisition settings may for example be automatically chosen by the mobile device, e.g., by the mobile device having the camera, used for capturing the image. Thus, as an example, the mobile device having the camera, e.g., a smartphone or a smartphone camera system, may automatically choose or set the image acquisition settings according to an ambient situation, e.g., according to an ambient lighting. Additionally or alternatively, one, more than one or even all of the image acquisition settings may be defined or set manually or via a predefined algorithm.

Step b) may further comprise locking the one or more image acquisition settings of the camera. In particular, the one or more image acquisition settings of the camera may remain locked at least until step e) has been carried out, specifically at least until the second image of the test field is captured. As used herein, the term “locking” is a broad term and is to be given its ordinary and customary meaning to a person of ordinary skill in the art and is not to be limited to a special or customized meaning. The term specifically may, without limitation, refer to a process of keeping an arbitrary configuration consistent, e.g., unchanged and/or unvaried. As an example, the locked configuration may remain constant for a period of time, specifically for a time between at least two events. Particularly, when locking a configuration, starting at a first event the configuration or setting may for example remain unchanged until at least one second event. Thus, the locking of the one or more image acquisition settings may be or may comprise keeping the image acquisition settings constant or unchanged for a period of time, specifically for the duration between carrying out step b) of the method until step e) of the method has been carried out.

Specifically, step b) may further comprise deriving at least one parameter pertaining to the one or more image acquisition settings. For example, the parameter pertaining to the one or more image acquisition settings may be or may comprise at least one numerical value referring to the one or more image acquisition settings.

In particular, the parameter pertaining to the one or more image acquisition settings may for example be stored in a database, specifically in a memory of the mobile device and/or the camera. Thus, the parameter pertaining to the one or more image acquisition settings may be stored within the mobile device, specifically within the memory of the mobile device having the camera, used for capturing the images in steps b) and e). Additionally or alternatively, however, the parameter pertaining to the one or more image acquisition settings may also be stored in an external memory or database, for example in a cloud. Specifically, the database may be stored in a data storage device, such as for example in a memory, such as a volatile or non-volatile memory, e.g., in a memory of the mobile device, on an external hard disk or hard drive, on an external computer or computer network, on a solid-state-drive or in a cloud.

As an example, the at least one parameter pertaining to the one or more image acquisition settings may be selected from the group consisting of: a shutter speed, specifically an exposure time; a focus distance; a color adjustment, such as a color saturation; a pixel correction, specifically a noise reduction.

Further, at least one of the at least one parameters pertaining to the one or more image acquisition settings may be configured to be corrected or set by at least one algorithm, for example in order to prevent overexposure, specifically of white areas within the image. As an example, the at least one algorithm may be configured for correcting the at least one of the at least one parameters in order to ensure a minimum amount of image quality, specifically of the images captured in steps b) and e). In particular, the algorithm may for example be stored and/or run on the mobile device. Specifically, the algorithm may be stored in a memory of the mobile device and/or may be loadable into a processor of the mobile device. Additionally or alternatively, the algorithm may be run on the mobile device, for example on a processor of the mobile device.

Step c) may specifically comprise one or both of:
prompting a user to apply a sample, specifically a drop, of bodily fluid to the test field of the optical test strip; or
prompting the user to confirm application of the sample of bodily fluid to the test field of the optical test strip.

Further, step d) may comprise waiting for at least 5 s. Thus, the predetermined minimum amount of time may for example be at least 5 s.

Further, step f) may comprise comparing the at least two images captured in steps b) and e), respectively. In particular, when comparing the at least two images at least one of a color difference and an intensity difference may be detected. Thus, step f) may particularly comprise detecting one or both of the color difference and the intensity difference, for example by comparing the images, e.g., the pixels of the images, captured in steps b) and e).

In particular, step f) may comprise determining a relative measurement value Rᵣₑₗ from one or both of a color or an intensity of the test field of the first image I_{TF_1} and a color and/or an intensity of the test field of the second image I_{TF_2} by using the following function:

$${R}_{rel} = \frac{{I}_{TF \_ 2}}{{I}_{TF \_ 1}} . \qquad ( 1 )$$

Specifically, the function may further comprise a proportionality factor.

Additionally or alternatively, step f) may comprise determining a twice relative measurement value R_{twice_rel} from a color and/or an intensity of the test field of the first image I_{TF_1}, a color and/or an intensity of the test field of the second image I_{TF_2}, a color and/or an intensity of a reference area, e.g., a white area, on the optical test strip of the first image I_{WF_1} and a color and/or an intensity of the same reference area on the optical test strip of the second image I_{WF_2}, by using the following functions:

$${R}_{twice\_rel} = \frac{{I}_{TF\_ 2} / {I}_{WF\_ 2}}{{I}_{TF\_ 1} / {I}_{WF\_ 1}} . \qquad ( 2 )$$

Specifically, the function may further comprise a proportionality factor.

The method may further comprise step g) evaluating at least one ambient lighting condition. Specifically, an illumination, for example an illumination of the test field, may be evaluated in step g). In particular, step g) may for example be performed at least once at one or more of the following points in time:
between step a) and step b),
between step b) and step c),
between steps e) and f), or
after step f).

In particular, the at least one ambient lighting condition may specifically be evaluated at least once before performing step b) and for example after performing step a). Additionally or alternatively, step g) may for example be performed at least once before performing step e).

Specifically, step g) may further comprise adapting the at least one ambient lighting condition by turning on or off at least one illumination source of the mobile device. The term “illumination source” as used herein is a broad term and is to be given its ordinary and customary meaning to a person of ordinary skill in the art and is not to be limited to a special or customized meaning. The term specifically may, without limitation, refer to an arbitrary device adapted to generate light for illuminating an object. For example, the illumination source of the mobile device may comprise at least one light-emitting diode integrated into the mobile device. In particular, the illumination source may be a backlighting of the mobile device. The mobile device may comprise further illumination devices such as at least one illumination source illuminating the display and/or the display may itself be designed as further illumination source itself. Thus, as an example, the at least one illumination source may be turned on in order to lighten or brighten the at least one ambient lighting condition. In particular, the at least one illumination source may be turned on when generating light, e.g., generating light from the at least one light-emitting diode or backlight of the mobile device, is required. The at least one illumination source may be turned off when no light generation from the illumination source is required.

In a further aspect, a computer program including computer-executable instructions for performing the method when the computer program is executed on a computer or computer network is disclosed. The computer program is configured for performing the method of performing an analytical measurement based on a color formation reaction as described above or as will be described in further detail below, when the computer program is executed on the computer or computer network, specifically on a processor of a mobile device. The computer program may specifically be configured for performing at least steps b), d), e), f) and optionally steps c) and/or g) of the method of performing an analytical measurement.

For possible definitions of terms and possible embodiments, reference may be made to the description given above or as described in further detail below.

Specifically, the computer program may be stored on a computer-readable data carrier. Thus, further disclosed and proposed herein is a data carrier having a data structure stored thereon, which, after loading into a computer or computer network, such as into a working memory or main memory of the computer or computer network, may execute the method according to one or more of the embodiments disclosed herein.

In a further aspect, a mobile device for performing an analytical measurement is disclosed. The mobile device has at least one camera. Further, the mobile device is configured for performing at least steps b), d), e) and f) of the method of performing an analytical measurement as described above or as will be described in further detail below.

Again, for possible definitions of terms and possible embodiments, reference may be made to the description given above or as described in further detail below.

Further, the mobile device may comprise at least one illumination source configured for adapting at least one ambient lighting condition.

In particular, the mobile device may comprise at least one processor being programmed for controlling at least one of steps b), d), e), and f), and optionally steps c) and/or g). The term “processor” as used herein is a broad term and is to be given its ordinary and customary meaning to a person of ordinary skill in the art and is not to be limited to a special or customized meaning. The term specifically may refer, without limitation, to an arbitrary electronic circuit configured for operating on data. In particular, the processor may be configured for performing operations on at least one database, such as on a memory, e.g., on a memory of the mobile device. As an example, the processor may specifically be or may comprise an integrated circuit (IC), such as an application-specific integrated circuit (ASIC) or a field programmable gate array (FPGA).

In a further aspect, a kit for performing an analytical measurement is disclosed. The kit comprises:
at least one mobile device as described above or as will be described in further detail below; and
at least one optical test strip having at least one test field.

Again, for possible definitions of terms and possible embodiments, reference may be made to the description given above or as described in further detail below.

The term “kit” as used herein is a broad term and is to be given its ordinary and customary meaning to a person of ordinary skill in the art and is not to be limited to a special or customized meaning. The term specifically may refer, without limitation, to an assembly of a plurality of components, wherein the components each may function and may be handled independently from each other, wherein the components of the kit may interact to perform a common function.

The methods, computer programs and devices as disclosed herein may provide a large number of advantages over known methods, computer programs and devices. Thus, in particular, this disclosure may provide for an improved reliability and accuracy of the analytical measurement compared to methods, computer programs and devices known from the art. Specifically, the reliability and accuracy may be improved by taking into account image processing steps of the mobile device, specifically any image processing steps performed by software, e.g., by an app, on the mobile device.

Further, this disclosure may simplify the process of performing the analytical measurement for a user. Specifically, the user may be able to perform the analytical measurement using a widely available mobile device, such as a smartphone, instead of a customized detector. In particular, this disclosure may be suitable for a vast number of mobile devices, such as a great variety of mobile phones or smartphones, specifically because this disclosure may for example be technically compatible with a majority of mobile devices, such that a technical implementation and/or use may be possible on a large number of mobile devices, such as mobile devices running at least one operating system, such as at least one operating system selected from the consisting of Android, Windows, iOS or Linux. Other operating systems may be feasible.

In addition, for this disclosure requirements for the camera system of the mobile device may be reduced compared to methods, computer programs and devices known from the art. In particular, the requirements for absolute color measurement or detection may for example be reduced due to a relative measurement, specifically a twice relative measurement, which may be used when performing the analytical measurement of this disclosure. Specifically, in this disclosure analyte measurement by using a relative color measurement, e.g., a relative intensity measurement, may for example be proposed. In particular, a first image of the optical test strip, e.g., of the test element, may be captured before application of the sample, specifically before the sample is applied to the test field of the optical test strip. When capturing the first image, the image acquisition settings may for example be chosen automatically by the mobile device, e.g., by the mobile device having the camera, used for capturing the image. Thus, as an example, the mobile device having the camera, e.g., a smartphone or a smartphone camera system, may automatically choose the image acquisition settings according to an ambient situation, e.g., according to an ambient lighting, when capturing the image, wherein overexposure of, for example, relevant parts of the optical test strip, e.g., of a white field, may be repressed. Additionally or alternatively, the image acquisition settings may be chosen manually or via a predefined algorithm. Subsequently, as an example, a capturing of a second image of the test element, e.g., after the sample has been applied to the test field, may specifically be performed using the same image acquisition settings. Thus, the image acquisition settings chosen when capturing the first image may be locked and/or stored, for example within the mobile device, until capturing the second image. Such functionality of locking and/or storing the image acquisition settings may specifically be supported by a vast number of mobile devices, e.g., by a large number of smartphones.

Summarizing and without excluding further possible embodiments, the following embodiments may be envisaged:

Embodiment 1: A method of performing an analytical measurement based on a color formation reaction in an optical test strip by using a mobile device having a camera, the method comprising:
a) providing an optical test strip having a test field without having a sample applied thereto;
b) capturing a first image of at least part of the test field of the optical test strip without having a sample applied thereto by using the camera with at least one image acquisition setting of the camera, specifically with a set of acquisition settings of the camera;
c) applying a sample, specifically a drop, of bodily fluid to the test field of the optical test strip;
d) waiting for a predetermined minimum amount of time;
e) capturing a second image of at least part of the test field of the optical test strip having the sample of bodily fluid applied thereto by using the camera with the one or more image acquisition settings of the camera, wherein the image acquisition settings of the camera are the same image acquisition settings of the camera as used in step b); and
f) determining an analytical measurement result value by using the first and the second image of the optical test field of the optical test strip, specifically by comparing the at least two images.

Embodiment 2: The method according to the preceding embodiment, wherein step b) further comprises locking the one or more image acquisition settings of the camera, wherein the one or more image acquisition settings of the camera remain locked at least until step e) has been carried out, specifically at least until the second image of the test field is captured.

Embodiment 3: The method according to any one of the preceding embodiments, wherein step b) further comprises deriving at least one parameter pertaining to the one or more image acquisition settings.

Embodiment 4: The method according to the preceding embodiment, wherein the parameter pertaining to the one or more image acquisition settings is stored in a database, specifically in a memory of the mobile device and/or of the camera.

Embodiment 5: The method according to the preceding embodiment, wherein the at least one parameter pertaining to the one or more image acquisition settings is selected from the group consisting of: a shutter speed, specifically an exposure time; a focus distance; a color adjustment, such as a color saturation; a pixel correction, specifically a noise reduction.

Embodiment 6: The method according to the preceding embodiment, wherein at least one of the at least one parameters pertaining to the one or more image acquisition settings is configured to be corrected or set by at least one algorithm, for example in order to prevent overexposure, specifically of white areas within the image, wherein the at least one algorithm is for example stored and/or run on the mobile device, specifically on a processor of the mobile device.

Embodiment 7: The method according to any one of the preceding embodiments, wherein step c) comprises one or both of:
prompting a user to apply the sample, specifically a drop, of bodily fluid to the test field of the optical test strip; or
prompting the user to confirm application of the sample of bodily fluid to the test field of the optical test strip.

Embodiment 8: The method according to any one of the preceding embodiments, wherein step d) comprises waiting for at least 5 s.

Embodiment 9: The method according to any one of the preceding embodiments, wherein step f) comprises comparing the at least two images captured in steps b) and e) respectively, wherein at least one of a color difference and an intensity difference is detected.

Embodiment 10: The method according to the preceding embodiment, wherein step f) comprises determining a relative measurement value Rᵣₑₗ from one or both of a color or an intensity of the test field of the first image I_{TF_1} and a color and/or intensity of the test field of the second image I_{TF_2} by using the following function:

$${R}_{rel} = \frac{{I}_{TF \_ 2}}{{I}_{TF \_ 1}} . \qquad ( 1 )$$

Embodiment 11: The method according to any one of the two preceding embodiments, wherein step f) comprises determining a twice relative measurement value R_{twice_rel} from a color and/or an intensity of the test field of the first image I_{TF_1}, a color and/or an intensity of the test field of the second image I_{TF_2}, a color and/or an intensity of a reference area, e.g., a white area, on the optical test strip of the first image I_{WF_1} and a color and/or an intensity of the same reference area on the optical test strip of the second image I_{WF_2}, by using the following functions:

$${R}_{twice\_rel} = \frac{{I}_{TF\_ 2} / {I}_{WF\_ 2}}{{I}_{TF\_ 1} / {I}_{WF\_ 1}} . \qquad ( 2 )$$

Embodiment 12: The method according to any one of the preceding embodiments, wherein the method further comprises
g) evaluating at least one ambient lighting condition,
   wherein step g) is performed at least once at one or more of the following points in time:
   between step a) and step b),
   between step b) and step c),
   between steps e) and f), or
   after step f).

Embodiment 13: The method according to the preceding embodiment, wherein step g) further comprises adapting the at least one ambient lighting condition by turning on or off at least one illumination source of the mobile device.

Embodiment 14: A computer program including computer-executable instructions for performing the method according to any one of the preceding embodiments when the computer program is executed on a computer or computer network, specifically on a processor of a mobile device.

Embodiment 15: A mobile device for performing an analytical measurement, the mobile device having at least one camera, the mobile device being configured for performing at least steps b), d), e) and f) of the method of performing an analytical measurement according to any one of the preceding embodiments referring to a method of performing an analytical measurement.

Embodiment 16: The mobile device according to the preceding embodiment, wherein the mobile device further comprises at least one illumination source configured for adapting at least one ambient lighting condition.

Embodiment 17: The mobile device according to any one of the two preceding embodiments, wherein the mobile device comprises at least one processor being programmed for controlling at least one of steps b), d), e), and f), and optionally c) and/or g).

Embodiment 18: A kit for performing an analytical measurement, the kit comprising:
at least one mobile device according to any one of the preceding embodiments referring to a mobile device; and
at least one optical test strip having at least one test field.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a perspective view of an embodiment of a kit and a mobile device;

FIGS. 2A and 2B show flow charts of embodiments of a method for performing an analytical measurement; and

FIGS. 3A and 3B show embodiments of diagrams indicating color signal counts recognizable by a camera over time when performing an analyte measurement.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

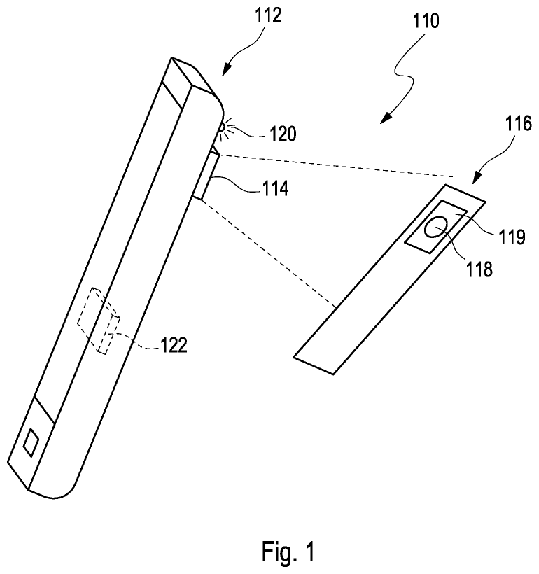

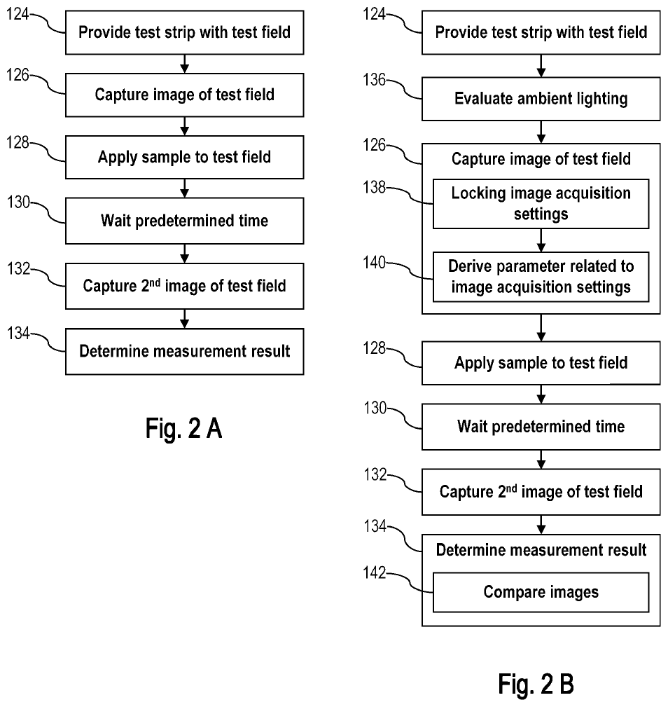

In FIG. 1 a kit 110 for performing analytical measurement is shown in a perspective view. The kit 110 comprising at least one mobile device 112 having at least one camera 114, the kit further comprising at least one optical test strip 116 having at least one test field 118. The optical test strip 116 may further have at least one white area 119. The mobile device 112 is configured for performing a method of performing an analytical measurement, as for example shown in FIGS. 2A and 2B. As illustrated in FIG. 1, the mobile device 112 may further comprise at least one illumination source 120. In particular, the mobile device 112 may comprise at least one processor 122 being programmed for controlling the steps of the method of performing an analytical measurement.

FIG. 2A shows an embodiment of a flow chart of a method of performing an analytical measurement based on a color formation reaction in an optical test strip 116 by using a mobile device 112 having a camera 114. The method comprises the following steps:
a) (indicated with reference number 124) providing an optical test 116 strip having a test field 118 without having a sample applied thereto;
b) (indicated with reference number 126) capturing at least one first image of at least part of the test field 118 of the optical test strip 116 without having a sample applied thereto by using the camera 114 with at least one image acquisition setting of the camera 114, specifically with a set of acquisition settings of the camera 114;
c) (indicated with reference number 128) applying one or both of a sample, specifically a drop, of bodily fluid to the test field 118 of the optical test strip 116;
d) (indicated with reference number 130) waiting for a predetermined minimum amount of time;
e) (indicated with reference number 132) capturing at least one second image of at least part of the test field of the optical test strip having the sample of bodily fluid applied thereto by using the camera with the one or more image acquisition settings of the camera 114, wherein the image acquisition settings of the camera are the same image acquisition settings of the camera 114 as used in step b); and
f) (indicated with reference number 134) determining an analytical measurement result value by using the first and the second image of the optical test field of the optical test strip, specifically by comparing the at least two images.

The mobile device 112 may in particular be configured for performing at least steps b), d), e) and f) of the method of performing an analytical measurement. In particular, the processor 122 may be programmed for controlling at least one of steps b), d), e) and f).

In FIG. 2B, a different embodiment of a flow chart of the method of performing an analytical measurement is shown. The method may further comprise step g) (indicated with reference number 136) evaluating at least one ambient lighting condition. In particular, step g) may, as illustrated in the figure, be performed at least once between step a) and step b). However, additionally or alternatively, step g) may be performed between step b) and c) and/or between step e) and step f) and/or after step f). The method, specifically step b) of the method, may further comprise at least one substep (indicated with reference number 138) of locking the one or more image acquisition settings of the camera 114. Specifically, the image acquisition settings or the set of image acquisition settings may remain locked until step e) of the method has been carried out. In particular, the image acquisition settings may remain locked at least until the second image of the test field has been captured. Additionally or alternatively, step b) may comprise at least one substep (indicated with reference number 140) of deriving at least one parameter pertaining to the one or more image acquisition settings. Further, step f) may comprise at least one substep (indicated with reference number 142) of comparing the at least two images captured in steps b) and e) respectively, in particular comparing the images with each other, wherein at least one of a color difference and an intensity difference may be detected. As an example, in step f) a relative measurement value Rᵣₑₗ 144 may be determined by using function (1) as described above. Additionally or alternatively, in step f) a twice relative measurement value R_{twice_rel} 146 may be determined by using function (2) as described above.

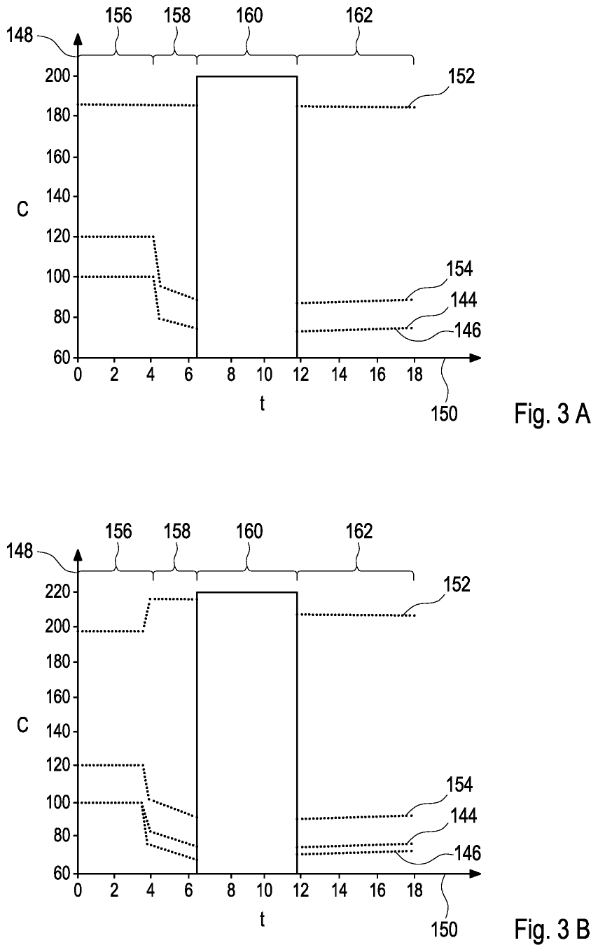

FIGS. 3A and 3B show embodiments of diagrams indicating color signal counts (C) recognizable by a camera 114, for example by the camera 114 of the mobile device 112, over time (t) when performing an analyte measurement. In particular, color signal counts for red color, e.g., R value 148, are shown over time measured in seconds 150. In the diagrams, measurement of samples with a blood glucose concentration of 50 mg/dl may be illustrated. Specifically, in the diagrams, color signal counts or R value for the white area 152 and the test field 154 of the optical test strip 116 are illustrated over time. Further, in the diagrams Rᵣₑₗ 144 and R_{twice_rel} 146 are shown, wherein Rᵣₑₗ 144 and R_{twice_rel} 146 may be determined by using functions (1) and (2) respectively.

As an example, in Figured 3A and 3B, performance of at least steps b), c), d) and e) of the method of performing an analytical measurement based on a color formation reaction may be illustrated. Thus, step b), in particular the capturing of the at least one first image, may be performed within a first time slot 156, wherein step c), specifically the application of a sample of bodily fluid to the test field 118, may be performed within a second time slot 158. Further, a third time slot 160 may as an example equal the predetermined minimum amount of time waited for in step d) of the method. Step e), in particular the capturing of the at least one second image, may be performed within a fourth time slot 162.

Specifically, in FIG. 3A, ambient light condition is constant. Thus, for example, the R value of the white area 152 may show a constant course over time, e.g., over the duration of the measurement. As illustrated in FIG. 3A, for constant ambient lighting conditions Rᵣₑₗ 144≈R_{twice_rel} 146. However, in FIG. 3B, ambient light condition changes. Thus, specifically the R value of the white area 152, illustrated in FIG. 3B, may change in the course of the measurement. Further, as illustrated in FIG. 3B, changing ambient light conditions may lead to Rᵣₑₗ 144 R_{twice_rel} 146. In particular, when determining Rᵣₑₗ 144, as an example, the R value of the test field 154 after sample application may be set in relation to the R value of the test field 154 without having a sample applied thereto, wherein the R value of the white area 152 may not be taken into account when determining Rᵣₑₗ 144. Thus, changing ambient lighting conditions indicated, for example, by the R value of the white area 152, specifically of the white field or reference field, may not be taken into account when determining Rᵣₑₗ 144. However, the R value of the white area 152 may be used for determining R_{twice_rel} 146. Thus, when determining of R_{twice_rel} 146 ambient lighting conditions may be taken into account. In particular, in case R_{twice_rel} 146 is used in step f) of the method, the determined analytical measurement result value may for example even be independent of changes in ambient lighting during performing of the method.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 kit
112 mobile device
114 camera
116 optical test strip
118 test field
119 white area
120 illumination source
122 processor
124 step a)
126 step b)
128 step c)
130 step d)
132 step e)
134 step f)
136 step g)
138 Substep
140 Substep
142 Substep
144 Rᵣₑₗ
146 R_{twice rel}
148 R value
150 Seconds
152 R value of white area
154 R value of test field
156 first time slot
158 second time slot
160 third time slot
162 fourth time slot

What is claimed is:

1. A method of performing an analytical measurement based on a color formation reaction in an optical test strip using a mobile device having a camera, the method comprising:
a) providing an optical test strip having a test field;
b) capturing a first image of at least part of the test field using the camera with an image acquisition setting of the camera;
c) applying a sample of body fluid to the test field;
d) waiting a predetermined minimum amount of time;
e) capturing a second image of at least part of the test field using the camera with the same image acquisition setting of the camera used in step b); and
f) determining an analytical measurement result value by using the first and the second images;
wherein step b) further comprises locking the image acquisition setting, and wherein the image acquisition setting remains locked at least until step e) has been performed.

2. The method according to claim 1, wherein step b) further comprises deriving a parameter pertaining to the image acquisition setting.

3. The method according to claim 2, wherein the parameter is stored in a database.

4. The method according to claim 3, wherein the parameter is selected from the group consisting of: a shutter speed; a focus distance; a color adjustment; a pixel correction.

5. The method according to claim 4, wherein the parameter is configured to be corrected or set by at least one algorithm.

6. The method according to claim 1, wherein step c) comprises one or both of:
(i) prompting a user to apply the sample of body fluid to the test field;
(ii) prompting the user to confirm application of the sample of body fluid to the test field.

7. The method according to claim 1, wherein step f) comprises comparing the images captured in steps b) and e), respectively, wherein at least one of a color difference and an intensity difference is detected.

8. The method according to claim 1, wherein the method further comprises:
g) evaluating at least one ambient lighting condition, wherein step g) is performed at least once at one or more of the following points in time:
between step a) and step b),
between step b) and step c),
between steps e) and f), and
after step f).

9. The method according to claim 8, wherein step g) further comprises adapting the ambient lighting condition by turning on or off at least one illumination source of the mobile device.

10. A non-transitory computer-readable medium having stored thereon computer-executable instructions for performing the method according to claim 1.

11. A mobile device for performing an analytical measurement, the mobile device comprising:
at least one camera;
a processor configured to perform steps b), d), e) and f) of claim 1.

12. The mobile device according to claim 11, further comprising an illumination source configured for adapting at least one ambient lighting condition.

13. A kit for performing an analytical measurement, the kit comprising:
a mobile device according to claim 11; and
an optical test strip having at least one test field.
